(19) United States
(12) Patent Application Publication
(10) Pub. No.: US 2005/0103330 A1
(43) Pub. Date: May 19, 2005

(54) Process of manufacturing drug delivery sprayheads

(76) Inventor: Gregor John McLennan Anderson, Ware (GB)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/432,204

(22) PCT Filed: Nov. 20, 2001
(86) PCT No.: PCT/EP01/13421

Related U.S. Application Data
(63) Continuation-in-part of application No. 09/717,876, filed on Nov. 21, 2000, now abandoned.

(51) Int. Cl. B21D 51/16; A61M 11/00

(52) U.S. Cl. 128/200.23; 29/890.09

(57) ABSTRACT

A process for manufacturing inhalation sprayheads comprising: (a) providing a metallic substrate having a thickness; (b) removing material from the metallic substrate to create one or more holes through the thickness, to create a holed metallic substrate possessing residual material upon said substrate; and (c) exposing the holed metallic substrate to an electrolyte and anodically eroding said residual material from the holed metallic substrate. Devices incorporating such sprayheads for the generation of small droplets are also described.

The present invention relates to the manufacture of atomization sprayheads used in the production of aerosol mists for medicinal inhalation purposes, and more particularly to the manufacture of piezoelectric sprayheads formed by laser drilling which deliver droplets less than 10 microns in size.

### BACKGROUND OF THE INVENTION

Piezoelectric spray generation devices for delivering respirable particles of liquid medicaments to a patient's pulmonary system have been disclosed in the prior art. Such atomization systems rely on sprayheads comprising a thin sheet of material that has one or more nozzles formed therein. The nozzles generate a fine mist of liquid medicament from a volume of a liquid solution or suspension of the medicament via piezoelectric vibration.

One way of achieving droplet formation, as described representatively disclosed in U.S. Pat. No. 5,299,739 (to Takahashi) the disclosure of which is herein by reference, is by extruding liquid droplets through the nozzles formed in a sprayhead by applying a brief pressure pulse that forces a small volume of the liquid out of the nozzle. The pressure pulse may be applied to the volume of liquid behind the nozzle in the sprayhead, or it may be caused by a contraction of the sprayhead against the liquid, causing a pressure increase in the liquid, thus, forcing droplets to be released through the nozzle. The droplet generated tends to be 1.5 to 2 times larger than the nozzle diameter in the sprayhead.

A second way of generating an aerosol mist from sprayhead coupled to an oscillating piezoelectric component is disclosed in U.S. Pat. No. 5,823,428 (to Humberstone), the disclosure of which is also incorporated by reference. '428 to Humberstone discloses that droplets may be formed as a result of the sprayhead being oscillated at a frequency that creates a waveform within a liquid. In such devices, the meniscus of the liquid is pinned within each given nozzle, between the inner and outer surfaces the sprayhead. The oscillation of the sprayhead creates a standing wave therein, causing droplets to be generated at the crests of the waveforms occurring in the meniscus of the liquid at positions corresponding to the nozzles. Droplets sheared from these waveform crests are released from the sprayhead. As discussed in this patent, and unlike the first class of oscillating nebulizing devices, this second class of devices is capable of delivering droplets whose diameters are not larger than the diameter of the nozzles through which they are generated. When the device operates with a “reverse taper” sprayhead ,i.e., one which employs tapered nozzles whose small diameter opening faces the liquid dose and whose larger diameter opening is on the outer face of the sprayhead.

Piezoelectric spray generating devices of these both classes are disclosed in WO 95/15822 and WO 94/22592 to The Technology Partnership, GB 2240494 and WO 94/09912 to Bespak PLC, U.S. Pat. No. 5,297,743 and to Toda, U.S. Pat. No. 5,487,379 and WO 92/11051 to 3M, and U.S. Pat. No. 5,299,739 to TDK Corporation, all of which are incorporated herein by reference.

To be pharmaceutically useful for treatment of lung conditions, droplets must be of a size capable of deposition in the appropriate area of a patient's lung. For topical delivery in the lung, the generated therapeutic particles must generally be between 1 and 10 microns in size, preferably less than 6 microns in size. For systemic delivery through the lung, the requirements are even more stringent, as the particles must generally be between 1 and 3 microns in size. To deliver particles of such size, sprayhead nozzles must be generated with diameters generally less than 20 microns, on the order of 1-10 microns, and preferably between 3 and 7 microns.

To allow the delivery a given dose volume of liquid medicament to a patient in a relatively short period of time, on the order of less than a few seconds, the sprayhead must also have a large number of such nozzles, to achieve a high rate of flow. Thus, a given sprayhead may have over 100 nozzles formed therein.

The nozzles of a sprayhead should be in relative alignment with each other, so the droplets generated from adjacent nozzles form discrete droplets, rather than in appropriately sized aggregates of material.

In all sprayheads must be generated with a high degree of precision.

Sprayheads have been manufactured from a variety of materials, including polymers, such as polyimides, plastics, ceramics, metals or metal alloys. Metallic or metallic alloy sprayheads have been proven to be particularly useful. Representative metal or metal alloy sprayheads include nickel, aluminum, copper, brass and stainless steel. These metallic sprayheads may be solid or plated with another metal, including gold.

As well described in the patent literature, sprayhead nozzles suitable for inhaled medicament delivery may be manufactured by a variety of processes. These include electro-deposition of metals, chemical etching of various materials, and high energy drilling, such as with laser drilling or ablation.

Laser drilling is particularly efficient for generating large numbers of sprayheads possessing nozzles between 1 and 20 microns in size, and particularly between 1 and 10 microns in size, in a repeatable and controllable fashion, with a high degree of uniform alignment. For example, U.S. Pat. No. 6,070,575 discloses that laser drilling is particularly useful in the creation of uniformly sized holes in materials such as polymers useful in creating aerosol sprayheads in non-oscillating systems. U.S. Pat. No. 5,811,019, discloses the use of laser drilling of materials including metals, for the manufacture of oscillating sprayheads for generating particles used in ink jet printers. Laser drilling of stainless steel nozzle plates used in ink jet print heads is disclosed in WO 90/04519. Such sprayheads may be applicable for respiratory drug delivery.

The ability of a sprayhead to generate respirable particles at relatively high flow rates may be greatly impaired if the holes are irregular in size, are not uniformly directed, are clogged, or are metallurgically modified during the drilling process. As stated above, nozzle diameter effects generated droplet size. It is desirable to have all droplets manufactured with approximately the same diameter to allow for appropriate deposition behavior in the lung. Therefore, all nozzles are desirably formed within a tight range of diameters.

The nozzles should be aligned accordingly to provide predictable droplet generation. If droplets are generated and impact each other, they may aggregate and form large particles not of respirable size. If the droplets are released from the sprayhead and impact the interior of a mouthpiece in a device, they may adhere to the walls, yielding undesirable device deposition. Hence nozzle alignment is important.

Spray generation is impaired if the surface of the sprayhead is rough, such as if it retains material intended to be removed during nozzle formation, and/or, particularly in the case of metallic sprayheads, is metallurgically or molecularly modified. Rough surfaces on these sprayheads may result in liquid adhering surface of the sprayhead, collecting around the exit side of the nozzles, and potentially blocking nozzle exits. In more severe cases, retained materials may completely block nozzles.

Drilling processes may result in marring or metallurgically altering the surface of the sprayhead, potentially rendering the sprayhead non-optimal for its intended purposes. On a molecular level, manufacturing may in some cases disrupt the crystalline, metallurgic or standard molecular properties of the sprayhead surface, especially in the case of laser drilled nozzles. In particular, laser welding has been acknowledged to effect the metallurgy of the drilled parts. Prior mechanisms for removing metallurgically altered regions have included mechanical drilling of the laser drilled regions, as disclosed in U.S. Pat. No. 3,696,054. The '054 method, however, is unsuitable for nozzles having very small diameters, i.e., in the region of 1-20 microns. Metallurgically altered regions of laser drilled components have also been removed using electrical discharge machining (EDM). EDM is material removal technique that uses electricity to remove metal by means of spark erosion in a dielectric fluid. As disclosed in U.S. Pat. No. 4,857,696, EDM operates by positioning the wire electrode of an electrical discharge machine adjacent a surface of the drilled hole. The wire electrode is advanced through the previously laser formed hole which is sized to provide an annular flow path for the unidirectional flow of an electrolyte. During the EDM process, the sparks from the electrode erode or vaporize the material to be removed in the previously drilled hole, and the particles produced are flushed from the part by the unidirectional flow of electrolyte. While EDM can be used to remove material from a laser drilled work piece, it tends to be complex and requires individual processing of each nozzle channel.

The present invention involves the creation of metal and metal alloy sprayheads for use in piezoelectric oscillating aerosol systems by removal of metal material to form nozzles in a sprayhead substrate. The invention is intended to achieve one or more of the following objectives:
1. The manufacture of sprayheads for piezoelectric nebulizing systems suitable for delivery of medicaments to a person
2. The manufacture of sprayheads with reduced residual material.
3. The manufacture of multi-orifice nozzles suitable for the generation of small particles.
4. The efficient manufacture of multiple nozzle sprayheads with smooth surfaced nozzles and reduced residual material.
5. The high volume, high-speed manufacture of sprayheads capable of generating small droplets.

### SUMMARY OF THE INVENTION

The current invention provides a high volume, reliable, low cost, effective method of removing residual material and/or metallurgically altered surfaces from sprayheads capable of generating droplets less than 10 microns, especially those formed by laser drilling.

The current invention relates a method of manufacturing sprayheads capable of delivering medicament of to the pulmonary system of a patient comprising the steps of:
a. Displacing a quantity of metal from a metal substrate to form a plurality of nozzles through said substrate having a diameter of less than 20 microns, to form one or more roughened surfaces on the surface of said substrate in the vicinity of said nozzles;
b. exposing said substrate to an electrolyte; and
c. electrically charging said metal substrate to dissolve said rough surfaces in said electrolyte.

The current invention also relates to sprayheads formed by such process, and medicinal inhalers incorporating such sprayheads.

### DETAILED DESCRIPTION

Laser drilling, as discussed above has been used to drill multiple nozzle sprayheads. As a by-product of laser drilling, ablated material may re-adhere onto the sprayhead. According to the present invention, these sprayheads can be treated to remove residual material by an electropolishing procedure. Electropolishing, also called electrolytic polishing, can be conducted by any means known in the art. Generally though, electropolishing includes coating the conductive metal substrate with an electrolytic material. The electrolytic material forms a polarizing film upon the surface of the substrate from which the sprayhead is fabricated. The coated substrate is exposed to an electric current, causing the conductive substrate material to anodically dissolve, thus removing the “high spots” of residual material on the conductive substrate leaving a smooth surface on the metallic surface of the conductive substrate material. Electropolishing may also remove a barrier laser of metallurgically modified material from the surface of the sprayhead. Removal of high spots and metallurgically modified surfaces allows more character standardization of all nozzles, and permits more precision in the ability to effectively pin a meniscus of liquid in the nozzle of the sprayhead in order to generate appropriately sized particles.

The resulting smooth surface may be employed in a piezoelectric oscillating sprayhead system for delivering small droplets of aerosolized medicament to the lung. It is believed to improve the flow rate and efficiency of the sprayhead, while assuring generation of a high percentage of drug droplets which are between 1 and 10 microns in size during use.

The metallic substrate is a conductive metal material suitable for use in a pharmaceutical sprayhead system, particularly inhalation devices, and most preferably those involving piezoelectric vibration. Preferred materials include metals and metal alloys, such as nickel, aluminum and stainless steel, either in coated or non-coated form.

The electrolytic material can be any solid, liquid or gas material suitable for forming an anodic highly polarized film on the metal that reacts with newly formed metal ions to dissolve them. Representative examples of suitable electropolishing solutions include phosphoric acid, sulfuric acid and cyanide baths. Practical electropolishing baths provide an anode film that is nearly saturated with the salt of the dissolving metal at a current density that maintains the conditions.

One of the advantages of the electropolishing process is that meshes, i.e., sprayheads, can be electropolished in bulk either all on one sheet, as they may be drilled in this way, or they may be stamped out once drilled and supported in carriers to go through electropolishing. The sheet format makes handling of the small delicate meshes much easier and more controllable from initial manufacture through to quality control. The bulk process allows a great number of sprayheads to be polished at the same time, yielding a relatively low cost, efficient manufacturing process. Accordingly, the process claimed in the instant invention may be carried out in the following fashion:

### EXAMPLE 1

A sheet of 25-micron thick sheet of stainless steel can be drilled to produce tapered nozzles having a minimum diameter of between 3 and 10 microns extending therethrough. The sheet is may then be degreased if required. It may then be placed into carrier/titanium jig/clamp/titanium barrel, and then exposed to an electrolyte chemical bath consisting of mixed acids at around 50 degrees centigrade. An electrical current can then be passed through the metallic sheet, providing it with a positive charge. The walls of the bath are negative. Any suitable amount of electricity can be used. It is believed that approximately 10 v current to approximately 1000 amps per square meter is optimal. Drilled components can be tumbled or the bath agitated to ensure contact. The control of electropolishing is primarily done by time. The time should be sufficient to remove the redeposited material that was displaced during the drilling process. The components may then be removed from the electrolytic bath, rinsed with a suitable liquid, for example tap water, then with a demineralized liquid, such as demineralized water and dried. With stainless steel it is preferred that the work piece be quickly immersed in an oxidization agent and then rinsed in -demineralized water prior to drying.

The process of the present invention thus is effective in creating sprayheads having aligned, appropriately sized, highly polished sprayheads for use in inhaler devices capable of producing small droplets. In such devices, the sprayhead formed by the claimed process is mounted in a nozzle assembly of the device. A volume of liquid medicament is placed in contact with one side of the sprayhead, and an oscillating piezoelectric is activated at a selected frequency to cause the discharge of droplets between 1 and 10 microns in size from the nozzles formed in the sprayhead.

The application of which this description and claims forms part may be used as a basis for priority in respect of any subsequent application. The claims of such subsequent application may be directed to any feature or combination of features described herein. They may take the form of product, composition, process, or use claims and may include by way of example and without limitation, the following claims:

1. A process for manufacturing inhalation sprayheads comprising:
a. providing a metallic substrate having a thickness;
b. removing material from said substrate to create one or more holes through the thickness, to create a holed metallic substrate possessing residual material upon said substrate;
c. exposing the holed metallic substrate to an electrolyte and anodically eroding said residual material from the holed metallic substrate.

2. The process of claim 1, wherein removal of material from the metallic substrate is accomplished by laser drilling.

3. The process of claim 1, wherein said electropolishing is performed only on the surface of the substrate upon which the residual material is present.

4. The process of claim 1, wherein said substrate provided comprises stainless steel.

5. The process of claim 1, wherein the hole generated is tapered, and wherein said tapered hole has a narrow opening on one side of said thickness of said metallic substrate, and a wider opening on the opposite side of said substrate, and said narrow opening has a diameter less than 10 microns in size.

6. The process of claim 5, wherein said substrate is stainless steel.

7. The process of claim 5, wherein said removal of material in said substrate is accomplished via laser drilling.

8. The process of claim 7 wherein said substrate is stainless steel.

9. A metallic sprayhead formed by the processes of claim 1 substantially free of residual material, positioned within a piezoelectricity vibrating inhaler capable of delivering droplets having a diameter less than 10 microns.

10. The metallic sprayhead of claim 9, wherein the sprayhead is stainless steel.

11. The metallic sprayhead of claim 9 wherein the holes are created by laser drilling.

12. The metallic sprayhead of claim 11 wherein the holes are tapered.
